(19) United States
(12) Patent Application Publication
(10) Pub. No.: US 2026/0355277 A1
(43) Pub. Date: Nov. 12, 2026

(54) Treatment Of Type 2 Diabetes With Wolfram Syndrome 1 (WFS1) Agonists

(71) Applicant: Regeneron Pharmaceuticals, Inc., Tarrytown, NY (US)

(72) Inventors: Moeen Riaz, Tarrytown, NY (US); Joshua Backman, Tarrytown, NY (US); Alan Shuldiner, Tarrytown, NY (US)

(21) Appl. No.: 19/644,189

(22) Filed: Apr. 10, 2026

Related U.S. Application Data
(60) Provisional application No. 63/787,111, filed on Apr. 11, 2025.

(51) Int. Cl. A61K 38/17; A61K 31/133; A61K 31/155; A61K 31/381; A61K 31/4045; A61K 31/428; A61K 31/445; A61K 31/48; A61K 31/4985; A61K 31/513; A61K 31/702; C12Q 1/6883

(52) U.S. Cl. CPC A61K 38/1709; A61K 31/133; A61K 31/155; A61K 31/381; A61K 31/4045; A61K 31/428; A61K 31/445; A61K 31/48; A61K 31/4985; A61K 31/513; A61K 31/702; C12Q 1/6883; C12Q 2600/156

(57) ABSTRACT

The present disclosure generally relates to the treatment of subjects having Type 2 diabetes or at risk of developing Type 2 diabetes by administering a Wolfram Syndrome 1 (WFS1) agonist to the subject, and to methods of identifying subjects having an increased risk of developing Type 2 diabetes.

### FIELD

The present disclosure generally relates to the treatment of subjects having Type 2 diabetes or at risk of developing Type 2 diabetes, by administering a Wolfram Syndrome 1 (WFS1) agonist to the subject, and to methods of identifying subjects having an increased risk of developing Type 2 diabetes.

### BACKGROUND

The global epidemic of Type 2 diabetes (T2D) is a major public health problem, as this disease is the fifth leading cause of death worldwide and a leading cause of morbidity, premature coronary heart disease, stroke, peripheral vascular disease, renal failure, and amputation. The number of individuals living with diabetes worldwide is predicted to increase from 366 million in 2011 to 552 million by 2030. Type 2 diabetes is a non-insulin-dependent diabetes that is characterized by hyperglycemia due to impaired insulin secretion and insulin resistance in target tissues. Type 2 diabetes is typically diagnosed after the age of 40 years and is caused by the combined action of genetic susceptibility and environmental factors. Type 2 diabetes is associated with obesity, and it is also a polygenic disease.

The Wolfram Syndrome 1 (WFS1) gene (also known as DFNA14, DFNA38, DFNA6, DIDMOAD, and WFS) encodes the wolframin protein. Wolframin is an 890-amino acid protein with nine transmembrane domains that localizes in the endoplasmic reticulum (ER) membrane and is involved in ER function and mitochondria Ca²⁺ transfer. A proper calcium balance is important for many different cellular functions, including cell-to-cell communication, the contraction of muscles, and protein processing. Wolframin is found in many different tissues, including pancreas, brain, heart, bones, muscles, lungs, liver, and kidneys. Wolframin is thought to play a role in protein folding and aid in the maintenance of endoplasmic reticulum function by regulating calcium levels. In the inner ear, wolframin may help maintain the proper levels of calcium ions or other charged particles that are essential for hearing.

### SUMMARY

The present disclosure provides methods of treating a subject having Type 2 diabetes, or at risk of developing Type 2 diabetes, the methods comprising administering a WFS1 agonist to the subject.

The present disclosure also provides methods of treating a subject having Type 2 diabetes or at risk of developing Type 2 diabetes by administering a Type 2 diabetes therapeutic agent and/or a WFS1 agonist to the subject, the methods comprising: determining or having determined whether the subject has a WFS1 variant nucleic acid molecule, by: obtaining or having obtained a biological sample from the subject; and performing or having performed a sequence analysis on the biological sample to determine if the subject has a genotype comprising a WFS1 variant nucleic acid molecule; and administering or continuing to administer the Type 2 diabetes therapeutic agent in an amount that is the same as or less than a standard dosage amount and/or administering a WFS1 agonist to a subject that is WFS1 reference or to a subject that is heterozygous for the WFS1 variant nucleic acid molecule; or administering or continuing to administer the Type 2 diabetes therapeutic agent in an amount that is the same as or less than a standard dosage amount and/or a WFS1 agonist to a subject that is homozygous for the WFS1 variant nucleic acid molecule; wherein the presence the WFS1 variant nucleic acid molecule indicates the subject has a decreased risk of developing Type 2 diabetes.

The present disclosure also provides methods of identifying a subject having an increased risk of developing Type 2 diabetes, the methods comprising: determining or having determined the presence or absence of a WFS1 variant nucleic acid molecule in a biological sample obtained from the subject; wherein: when the subject is WFS1 reference, then the subject has an increased risk of developing Type 2 diabetes; and when the subject is heterozygous or homozygous for the WFS1 variant nucleic acid molecule, then the subject has a decreased risk of developing Type 2 diabetes.

The present disclosure also provides Type 2 diabetes therapeutic agents for use in the treatment or prevention of Type 2 diabetes in a subject having a WFS1 variant nucleic acid molecule.

The present disclosure also provides WFS1 agonist for use in the treatment or prevention of Type 2 diabetes in a subject that is WFS1 reference or in a subject that is heterozygous for a WFS1 variant nucleic acid molecule.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows representative results of exome-wide and genome-wide association studies showing that missense variant 4:6302175:G:A (p.Glu794Lys) in WFS1 is associated with protection from Type 2 diabetes in South Asians; AAF: Alternate allele Freq, Cases: refers to T2D, controls: correspond to people with no T2D. Ref/Het/Hom: reference/heterozygous/homozygous genotype of variant 4:6302175:G:A, geno freeze: study cohorts included in the analysis, OR (95% CI): odd ratio ninety five percent confidence intervals.

FIG. 2 shows representative results of exome-wide and genome-wide association studies showing WFS1 p.Glu794Lys is associated with lower HbA1c.

FIG. 3 shows representative results of exome-wide and genome-wide association studies showing WFS1 p.Glu794Lys is associated with lower glucose in SAS and trans-ethnic meta.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by +10% and remain within the scope of the disclosed embodiments.

As used herein, the term “comprising” may be replaced with “consisting” or “consisting essentially of” in particular embodiments as desired.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horses, cows, and pigs), companion animals (such as, for example, dogs and cats), laboratory animals (such as, for example, mice, rats, and rabbits), and non-human primates. In some embodiments, the subject is a human. In some embodiments, the human is a patient under the care of a physician. In some embodiments, the subject does not have Wolfram Syndrome. In some embodiments, the subject is in need of treating Type 2 diabetes (i.e., has been diagnosed as having Type 2 diabetes or at risk of developing Type 2 diabetes; “in need thereof”).

Common variants in WFS1 are associated with increased risk of Type 2 diabetes (p 1.58e-08, effect 1.06), pLOF_missense (5/5) mask associated with Type 2 diabetes (p 2.8e-05. effect 1.09). However, it has been surprisingly observed in accordance with the present disclosure that a rare WFS1 missense variant nucleic acid molecule (whether the variant is homozygous or heterozygous in a particular subject) associates with a decreased risk of developing Type 2 diabetes. In particular, an exome/genome wide association study of 32,752 Type 2 diabetes cases and 99,293 controls in South Asians (SA) (CNCD, CAMB, UCLA, SAB and UKB cohorts) adjusted for age, sex and genetic principal components was carried out. A protective association of the missense variant 4:6302175:G: A (p.Glu794Lys) in WFS1 (effect 0.56, p=2.7e-08) was observed with Type 2 diabetes. Position 794 of human WFS1 polypeptide is present within Exon 8. The frequency of p.Glu794Lys was markedly enriched in SA (AAF=0.003) compared to EURs (AAF=9.841e-6). It is believed that the WFS1 rare missense variant nucleic acid molecule p.Glu794Lys has not been associated with increased risk of Type 2 diabetes in humans. Therefore, subjects that are WFS1 reference or heterozygous for a WFS1 variant nucleic acid molecule may be treated with a WFS1 agonist such that Type 2 diabetes is inhibited or prevented, the symptoms thereof are reduced or prevented, development of symptoms is repressed or prevented, the progression of prediabetes to Type 2 diabetes is slowed or prevented, and/or progressive beta cell dysfunction is slowed or prevented. It is also believed that such subjects having Type 2 diabetes may further be treated with one or more Type 2 diabetes therapeutic agents. In addition, the present disclosure provides methods of leveraging the presence or absence of WFS1 variant nucleic acid molecules in subjects to identify or stratify risk is such subjects of developing Type 2 diabetes, or to diagnose subjects as having an increased risk of developing Type 2 diabetes.

For purposes of the present disclosure, any particular subject, such as a human, can be categorized as having one of three WFS1 genotypes: i) WFS1 reference; ii) heterozygous for a WFS1 variant nucleic acid molecule; or iii) homozygous for a WFS1 variant nucleic acid molecule. A subject is WFS1 reference when the subject does not have a copy of a WFS1 variant nucleic acid molecule. A subject is heterozygous for a WFS1 variant nucleic acid molecule when the subject has a single copy of a WFS1 variant nucleic acid molecule. A subject is homozygous for a WFS1 variant nucleic acid molecule when the subject has two copies of a WFS1 variant nucleic acid molecule.

In any of the embodiments described herein, the WFS1 variant nucleic acid molecule can be any nucleic acid molecule (such as, a genomic nucleic acid molecule, an mRNA molecule, or a cDNA molecule produced from an mRNA molecule) encoding a WFS1 variant polypeptide having a partial gain-of-function, a complete gain-of-function, a predicted partial gain-of-function, or a predicted complete gain-of-function (collectively, a “gain-of-function”). A subject who has a WFS1 polypeptide having a partial gain-of-function (or predicted partial gain-of-function) is hypomorphic for WFS1. In some embodiments, the WFS1 variant nucleic acid molecule may result in increased expression or activity of WFS1 mRNA or polypeptide. In some embodiments, the WFS1 variant nucleic acid molecule may be associated with an increased in vitro response to WFS1 ligands compared to reference WFS1. In some embodiments, the WFS1 variant nucleic acid molecule is a splice-site variant, a stop-gain variant, a start-loss variant, a stop-loss variant, a frameshift variant, or an in-frame indel variant. In some embodiments, the WFS1 variant nucleic acid molecule is a missense variant nucleic acid molecule. In some embodiments, the WFS1 variant nucleic acid molecule comprises a single nucleotide polymorphism (SNP). In some embodiments, the WFS1 variant nucleic acid molecule comprises a variation in a coding region. In some embodiments, the WFS1 variant nucleic acid molecule does not comprise a variation in a non-coding region, except for a splice acceptor region (two bases before the start of any exon except the first). In some embodiments, the WFS1 variant nucleic acid molecule is a variant that is predicted to be enhancing to the protein function (and hence, in this case, protective to the human) by in vitro prediction algorithms such as Polyphen, SIFT, or similar algorithms. In some embodiments, the WFS1 variant nucleic acid molecule is a variant that causes or is predicted to cause a nonsynonymous amino acid substitution in a WFS1 nucleic acid molecule and whose allele frequency is less than 1/100 alleles in the population from which the subject is selected. In some embodiments, the WFS1 variant nucleic acid molecule is a rare missense variant (allele frequency<0.1%; or 1 in 1,000 alleles), or a splice-site, stop-gain, start-loss, stop-loss, frameshift, or in-frame indel.

In any of the embodiments described herein, the WFS1 variant genomic nucleic acid molecule may include one or more variations at any of the positions of human chromosome 4 (i.e., positions 6,269,850-6,303,265) using the nucleotide sequence of the WFS1 reference genomic nucleic acid molecule in the GRCh38/hg38 human genome assembly (see, ENSG00000109501.15, ENST00000226760.5, UniProt O76024, RefSeq NM_006005.3, annotated in the Ensembl database (URL: world wide web at “useast.ensembl.org/Homo_sapiens/Gene/Summary?g=ENSG00000109501;r=4:6269849-6303265;transcript=ENST00000226760.5”)) as a reference sequence. The sequences provided in this transcript for the WFS1 genomic nucleic acid molecule is only an exemplary sequence. Other sequences for the WFS1 genomic nucleic acid molecule are also possible. In any of the embodiments described herein, the WFS1 variant nucleic acid molecule may comprise any one or more of the following genetic variations in the genomic nucleic acid molecule (referring to the chromosome: positions set forth in the GRCh38/hg38 human genome assembly): 4:6302175:G:A (Glu794Lys), or an mRNA molecule produced therefrom, or a cDNA molecule produced from the mRNA molecule.

For subjects that are genotyped or determined to be WFS1 reference, such subjects have an increased risk of developing Type 2 diabetes compared to subjects that are heterozygous or homozygous for a WFS1 variant nucleic acid molecule. For subjects that are genotyped or determined to be either WFS1 reference or heterozygous for a WFS1 variant nucleic acid molecule, such subjects can be treated with a WFS1 agonist.

In any of the embodiments described herein, the subject in whom Type 2 diabetes is prevented by administering a WFS1 agonist may be anyone at risk for developing Type 2 diabetes including, but not limited to, subjects with a genetic predisposition for developing Type 2 diabetes. Additional risk factors for Type 2 diabetes include, but are not limited to, age (particularly after the age of 45), overweight, fat distribution, inactivity, race and ethnicity, prediabetes, and gestational diabetes. In any of the embodiments described herein, the methods can be used to improve Type 2 diabetes.

In any of the embodiments described herein, the WFS1 predicted gain-of-function polypeptide can be any WFS1 polypeptide having a partial gain-of-function, a complete gain-of-function, a predicted partial gain-of-function, or a predicted complete gain-of-function. In some embodiments, the WFS1 predicted gain-of-function polypeptide comprises WFS1 Glu794Lys.

Any one or more (i.e., any combination) of the WFS1 variant nucleic acid molecules described herein can be used within any of the methods described herein to determine whether a subject has an increased or decreased risk of developing Type 2 diabetes. The combinations of particular variants can form a mask used for statistical analysis of the particular association of WFS1 and an increased or decreased risk of developing Type 2 diabetes. In some embodiments, the mask used for statistical analysis of the particular association of WFS1 and an increased or decreased risk of developing Type 2 diabetes can exclude any one or more of these WFS1 variant nucleic acid molecules described herein.

In any of the embodiments described herein, the subject can have Type 2 diabetes. In any of the embodiments described herein, the subject can be at risk of developing Type 2 diabetes. In any of the embodiments described herein, the subject can be at any stage of Type 2 diabetes including insulin resistance and prediabetes.

In any of the embodiments described herein, the subject can be South Asian.

In any of the embodiments described herein, the methods can be used to treat a complication or co-morbidity of Type 2 diabetes or reduce the risk of developing the same. Complications and co-morbidities of Type 2 diabetes include, but are not limited to, eye problems (including cataracts and light sensitivity), skin infections and sores, nerve damage (pain, tingling, and numbness), kidney damage, heart disease, and stroke.

The present disclosure provides methods of treating a subject having Type 2 diabetes or at risk of developing Type 2 diabetes, the methods comprising administering a WFS1 agonist to the subject. The WFS1 agonist can be any one of the WFS1 agonists described herein.

The present disclosure also provides methods of slowing or preventing the progression of prediabetes to Type 2 diabetes, the methods comprising administering a WFS1 agonist to the subject. The WFS1 agonist can be any one of the WFS1 agonists described herein.

The present disclosure also provides methods of slowing or preventing progressive beta cell dysfunction, the methods comprising administering a WFS1 agonist to the subject. The WFS1 agonist can be any one of the WFS1 agonists described herein.

In some embodiments, the methods further comprise detecting the presence or absence of a WFS1 variant nucleic acid molecule encoding a WFS1 variant gain-of-function polypeptide in a biological sample from the subject. The WFS1 variant nucleic acid molecule can be any of the WFS1 variant nucleic acid molecules disclosed herein.

In some embodiments, the subject is also administered a Type 2 diabetes therapeutic agent. In some embodiments, the methods further comprise administering the Type 2 diabetes therapeutic agent in an amount that is the same as or less than a standard dosage amount to the subject when the WFS1 variant nucleic acid molecule is absent from the biological sample (i.e., the subject is WFS1 reference). In some embodiments, the methods further comprise administering the Type 2 diabetes therapeutic agent in an amount that is the same as or less than a standard dosage amount to the subject when the subject is heterozygous for the WFS1 variant nucleic acid molecule.

In some embodiments, the methods further comprise detecting the presence or absence of a WFS1 variant polypeptide in a biological sample from the subject. In some embodiments, when the subject does not have the WFS1 variant polypeptide, the subject is administered a Type 2 diabetes therapeutic agent in an amount that is the same as or less than a standard dosage amount. In some embodiments, when the subject has the WFS1 variant polypeptide, the subject is administered a Type 2 diabetes therapeutic agent in standard dosage amount.

In some embodiments, the methods further comprise detecting the presence or absence of an increase in the expression of a WFS1 variant mRNA or variant polypeptide in a biological sample from the subject. In some embodiments, when the subject does not have an increase in the expression of the WFS1 variant mRNA or variant polypeptide, the subject is administered a Type 2 diabetes therapeutic agent in an amount that is the same as or less than a standard dosage amount. In some embodiments, when the subject has an increase in the expression of the WFS1 variant mRNA or variant polypeptide, the subject is administered a Type 2 diabetes therapeutic agent in a standard dosage amount.

The present disclosure also provides methods of treating a subject having Type 2 diabetes or at risk of developing Type 2 diabetes by administering a Type 2 diabetes therapeutic agent and/or a WFS1 agonist to the subject. The methods comprise determining or having determined whether the subject has a WFS1 variant nucleic acid molecule encoding a WFS1 variant gain-of-function polypeptide by obtaining or having obtained a biological sample from the subject, and performing or having performed a sequence analysis on the biological sample to determine if the subject has a genotype comprising the WFS1 variant nucleic acid molecule. In some embodiments, the methods further comprise administering or continuing to administer the Type 2 diabetes therapeutic agent in an amount that is the same as or less than a standard dosage amount and/or the WFS1 agonist to a subject that is WFS1 reference. In some embodiments, the methods further comprise administering or continuing to administer the Type 2 diabetes therapeutic agent in an amount that is the same as or less than a standard dosage amount and the WFS1 agonist to a subject that is WFS1 reference. In some embodiments, the methods further comprise administering or continuing to administer the Type 2 diabetes therapeutic agent in an amount that is the same as or less than a standard dosage amount and/or the WFS1 agonist to a subject that is heterozygous for the WFS1 variant nucleic acid molecule. In some embodiments, the methods further comprise administering or continuing to administer the Type 2 diabetes therapeutic agent in an amount that is the same as or less than a standard dosage amount and the WFS1 agonist to a subject that is heterozygous for the WFS1 variant nucleic acid molecule. In some embodiments, the methods further comprise administering or continuing to administer the Type 2 diabetes therapeutic agent in an amount that is the same as or less than a standard dosage amount and/or a WFS1 agonist to a subject that is homozygous for the WFS1 variant nucleic acid molecule. In some embodiments, the methods further comprise administering or continuing to administer the Type 2 diabetes therapeutic agent in an amount that is the same as or less than a standard dosage amount and a WFS1 agonist to a subject that is homozygous for the WFS1 variant nucleic acid molecule. The presence of the WFS1 variant nucleic acid molecule indicates the subject has a decreased risk of developing Type 2 diabetes.

The present disclosure also provides methods of slowing or preventing the progression of prediabetes to Type 2 diabetes by administering a WFS1 agonist or a combination of a WFS1 agonist and a Type 2 diabetes therapeutic agent to the subject. The methods comprise determining or having determined whether the subject has a WFS1 variant nucleic acid molecule encoding a WFS1 variant gain-of-function polypeptide by obtaining or having obtained a biological sample from the subject, and performing or having performed a sequence analysis on the biological sample to determine if the subject has a genotype comprising the WFS1 variant nucleic acid molecule. In some embodiments, the methods further comprise administering or continuing to administer a WFS1 agonist or a combination of a WFS1 agonist and a Type 2 diabetes therapeutic agent to a subject that is WFS1 reference. In some embodiments, the methods further comprise administering or continuing to administer a WFS1 agonist or a combination of a WFS1 agonist and a Type 2 diabetes therapeutic agent to a subject that is heterozygous for the WFS1 variant nucleic acid molecule. In some embodiments, the methods further comprise administering or continuing to administer a WFS1 agonist or a combination of a WFS1 agonist and a Type 2 diabetes therapeutic agent to a subject that is homozygous for the WFS1 variant nucleic acid molecule. The presence of the WFS1 variant nucleic acid molecule indicates the subject has an increased odds of slowing or preventing the progression of prediabetes to Type 2 diabetes.

The present disclosure also provides methods of slowing or preventing progressive beta cell dysfunction by administering a WFS1 agonist or a combination of a WFS1 agonist and a Type 2 diabetes therapeutic agent to the subject. The methods comprise determining or having determined whether the subject has a WFS1 variant nucleic acid molecule encoding a WFS1 variant gain-of-function polypeptide by obtaining or having obtained a biological sample from the subject, and performing or having performed a sequence analysis on the biological sample to determine if the subject has a genotype comprising the WFS1 variant nucleic acid molecule. In some embodiments, the methods further comprise administering or continuing to administer a WFS1 agonist or a combination of a WFS1 agonist and a Type 2 diabetes therapeutic agent to a subject that is WFS1 reference. In some embodiments, the methods further comprise administering or continuing to administer a WFS1 agonist or a combination of a WFS1 agonist and a Type 2 diabetes therapeutic agent to a subject that is heterozygous for the WFS1 variant nucleic acid molecule. In some embodiments, the methods further comprise administering or continuing to administer a WFS1 agonist or a combination of a WFS1 agonist and a Type 2 diabetes therapeutic agent to a subject that is homozygous for the WFS1 variant nucleic acid molecule. The presence of the WFS1 variant nucleic acid molecule indicates the subject has an increased odds of slowing or preventing progressive beta cell dysfunction.

The present disclosure also provides methods of preventing a subject from developing Type 2 diabetes by administering a Type 2 diabetes therapeutic agent and/or a WFS1 agonist to the subject. The methods comprise determining or having determined whether the subject has a WFS1 variant nucleic acid molecule encoding a WFS1 variant gain-of-function polypeptide by obtaining or having obtained a biological sample from the subject, and performing or having performed a sequence analysis on the biological sample to determine if the subject has a genotype comprising the WFS1 variant nucleic acid molecule. In some embodiments, the methods further comprise administering or continuing to administer the Type 2 diabetes therapeutic agent in an amount that is the same as or less than a standard dosage amount and/or the WFS1 agonist to a subject that is WFS1 reference. In some embodiments, the methods further comprise administering or continuing to administer the Type 2 diabetes therapeutic agent in an amount that is the same as or less than a standard dosage amount and the WFS1 agonist to a subject that is WFS1 reference. In some embodiments, the methods further comprise administering or continuing to administer the Type 2 diabetes therapeutic agent in an amount that is the same as or less than a standard dosage amount and/or the WFS1 agonist to a subject that is heterozygous for the WFS1 variant nucleic acid molecule. In some embodiments, the methods further comprise administering or continuing to administer the Type 2 diabetes therapeutic agent in an amount that is the same as or less than a standard dosage amount and the WFS1 agonist to a subject that is heterozygous for the WFS1 variant nucleic acid molecule. In some embodiments, the methods further comprise administering or continuing to administer the Type 2 diabetes therapeutic agent in an amount that is the same as or less than a standard dosage amount and/or a WFS1 agonist to a subject that is homozygous for the WFS1 variant nucleic acid molecule. In some embodiments, the methods further comprise administering or continuing to administer the Type 2 diabetes therapeutic agent in an amount that is the same as or less than a standard dosage amount and a WFS1 agonist to a subject that is homozygous for the WFS1 variant nucleic acid molecule. The presence of the WFS1 variant nucleic acid molecule indicates the subject has a decreased risk of developing Type 2 diabetes.

Detecting the presence or absence of a WFS1 variant nucleic acid molecule in a biological sample from a subject and/or determining whether a subject has a WFS1 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

The present disclosure also provides methods of treating a subject having Type 2 diabetes or at risk of developing Type 2 diabetes by administering a Type 2 diabetes therapeutic agent and/or a WFS1 agonist to the subject. The methods comprise determining whether the subject has a WFS1 variant gain-of-function polypeptide by obtaining or having obtained a biological sample from the subject and performing or having performed an assay on the biological sample to determine if the subject has the WFS1 variant polypeptide. In some embodiments, when the subject does not have the WFS1 variant polypeptide, the subject is administered the Type 2 diabetes therapeutic agent in an amount that is the same as or less than a standard dosage amount and/or a WFS1 agonist. In some embodiments, when the subject does not have the WFS1 variant polypeptide, the subject is administered the Type 2 diabetes therapeutic agent in an amount that is the same as or less than a standard dosage amount and a WFS1 agonist. In some embodiments, when the subject has the WFS1 variant polypeptide, the subject is administered the Type 2 diabetes therapeutic agent in a standard dosage amount. The presence of the WFS1 variant gain-of-function polypeptide indicates the subject has a decreased risk of developing Type 2 diabetes. In some embodiments, the subject has a WFS1 variant gain-of-function polypeptide. In some embodiments, the subject does not have a WFS1 variant gain-of-function polypeptide.

The present disclosure also provides methods of preventing a subject from developing Type 2 diabetes by administering a Type 2 diabetes therapeutic agent and/or a WFS1 agonist to the subject. The methods comprise determining whether the subject has a WFS1 variant gain-of-function polypeptide by obtaining or having obtained a biological sample from the subject and performing or having performed an assay on the biological sample to determine if the subject has the WFS1 variant gain-of-function polypeptide. In some embodiments, when the subject does not have the WFS1 variant gain-of-function polypeptide, the subject is administered the Type 2 diabetes therapeutic agent in an amount that is the same as or less than a standard dosage amount and/or a WFS1 agonist. In some embodiments, when the subject does not have the WFS1 variant gain-of-function polypeptide, the subject is administered the Type 2 diabetes therapeutic agent in an amount that is the same as or less than a standard dosage amount and a WFS1 agonist. When the subject has the WFS1 variant gain-of-function polypeptide, the subject is administered the Type 2 diabetes therapeutic agent in a standard dosage amount. The presence of the WFS1 variant gain-of-function polypeptide indicates the subject has a decreased risk of developing Type 2 diabetes. In some embodiments, the subject has a WFS1 variant gain-of-function polypeptide. In some embodiments, the subject does not have a WFS1 variant gain-of-function polypeptide.

Detecting the presence or absence of a WFS1 variant gain-of-function polypeptide in a biological sample from a subject and/or determining whether a subject has a WFS1 variant gain-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the polypeptide can be present within a cell obtained from the subject.

The present disclosure also provides methods of treating a subject having Type 2 diabetes or at risk of developing Type 2 diabetes by administering a Type 2 diabetes therapeutic agent and/or a WFS1 agonist to the subject. The methods comprise determining whether the subject has an increase in the expression of a WFS1 variant mRNA or polypeptide by obtaining or having obtained a biological sample from the subject, and performing or having performed an assay on the biological sample to determine if the subject has an increase in the expression of the WFS1 variant mRNA or variant polypeptide. In some embodiments where the subject does not have an increase in the expression of the WFS1 variant mRNA or variant polypeptide, the methods further comprise administering or continuing to administer the Type 2 diabetes therapeutic agent in an amount that is the same as or less than a standard dosage amount and/or the WFS1 agonist to the subject. In some embodiments where the subject does not have an increase in the expression of the WFS1 variant mRNA or variant polypeptide, the methods further comprise administering or continuing to administer the Type 2 diabetes therapeutic agent in an amount that is the same as or less than a standard dosage amount and the WFS1 agonist to the subject. In some embodiments where the subject has an increase in the expression of the WFS1 variant mRNA or variant polypeptide, the methods further comprise administering or continuing to administer the Type 2 diabetes therapeutic agent in a standard dosage amount to the subject. The presence of an increase in the expression of the WFS1 variant mRNA or variant polypeptide indicates the subject has a decreased risk of developing Type 2 diabetes. In some embodiments, the subject has an increase in the expression of the WFS1 variant mRNA or variant polypeptide. In some embodiments, the subject does not have an increase in the expression of the WFS1 variant mRNA or variant polypeptide.

The present disclosure also provides methods of preventing a subject from developing Type 2 diabetes by administering a Type 2 diabetes therapeutic agent and/or a WFS1 agonist to the subject. The methods comprise determining whether the subject has an increase in the expression of a WFS1 variant mRNA or polypeptide by obtaining or having obtained a biological sample from the subject, and performing or having performed an assay on the biological sample to determine if the subject has an increase in the expression of the WFS1 variant mRNA or variant polypeptide. In some embodiments where the subject does not have an increase in the expression of the WFS1 variant mRNA or variant polypeptide, the methods further comprise administering or continuing to administer the Type 2 diabetes therapeutic agent in an amount that is the same as or less than a standard dosage amount and/or the WFS1 agonist to the subject. In some embodiments where the subject does not have an increase in the expression of the WFS1 variant mRNA or variant polypeptide, the methods further comprise administering or continuing to administer the Type 2 diabetes therapeutic agent in an amount that is the same as or less than a standard dosage amount and the WFS1 agonist to the subject. In embodiments where the subject has an increase in the expression of the WFS1 variant mRNA or variant polypeptide, the methods further comprise administering or continuing to administer the Type 2 diabetes therapeutic agent in a standard dosage amount to the subject. The presence of an increase in the expression of the WFS1 variant mRNA or variant polypeptide indicates the subject has a decreased risk of developing Type 2 diabetes. In some embodiments, the subject has an increase in the expression of the WFS1 variant mRNA or variant polypeptide. In some embodiments, the subject does not have an increase in the expression of the WFS1 variant mRNA or variant polypeptide.

Detecting a decrease in the expression of a WFS1 variant mRNA or variant polypeptide can be carried out by a variety of known methods. In some embodiments, these methods can be carried out in vitro. In some embodiments, these methods can be carried out in situ. In some embodiments, these methods can be carried out in vivo. In any of these embodiments, the mRNA or polypeptide can be present within a cell obtained from the subject.

In any of the embodiments described herein, the subject can have insulin resistance or prediabetes.

In any of the embodiments described herein, the subject is WFS1 reference. In any of the embodiments described herein, the subject can comprise a WFS1 variant nucleic acid molecule encoding a WFS1 variant gain-of-function polypeptide. In any of the embodiments described herein, the subject is heterozygous for a WFS1 variant nucleic acid molecule encoding a WFS1 variant gain-of-function polypeptide. In any of the embodiments described herein, the subject is homozygous for a WFS1 variant nucleic acid molecule encoding a WFS1 variant gain-of-function polypeptide.

In any of the embodiments described herein, the WFS1 variant nucleic acid molecule comprises a splice-site variant, a stop-gain variant, a start-loss variant, a stop-loss variant, a frameshift variant, a missense variant, or an in-frame indel variant. In some embodiments, the WFS1 variant nucleic acid molecule encodes WFS1 Glu794Lys polypeptide. In some embodiments, the WFS1 variant nucleic acid molecule is a WFS1 variant genomic nucleic acid molecule that comprises the genetic variation 4:6302175:G:A, or is an mRNA molecule produced therefrom, or is a cDNA molecule produced from the mRNA molecule.

In some embodiments, the WFS1 agonist comprises wolframin protein.

The present disclosure also provides compositions comprising any one or more of the WFS1 agonists. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the WFS1 agonist comprises an antibody, or antigen-binding fragment thereof. In some embodiments, the antibody, or antigen-binding fragment thereof, binds specifically to human WFS1. In some embodiments, the antibody is a fully human monoclonal antibody (mAb), or antigen-binding fragment thereof, that specifically binds and increases at least one activity of WFS1, in particular, human WFS1. The antibodies can be full-length (for example, an IgG1 or IgG4 antibody) or may comprise only an antigen-binding portion (for example, a Fab, F(ab′)₂ or scFv fragment), and may be modified to affect functionality.

In some embodiments, the antibody or antigen-binding fragment thereof specifically binds to WFS1 with an equilibrium dissociation constant (K_{D}) of about 7 nM or less, about 6 nM or less, about 5 nM or less, about 4 nM or less, about 3 nM or less, about 2 nM or less, or about 1 nM or less, as measured by surface plasmon resonance assay (for example, BIACORE™). In some embodiments, the antibody exhibits a K_{D} of about 800 pM or less, about 700 pM or less; about 600 pM or less; about 500 pM or less; about 400 pM or less; about 300 pM or less; about 200 pM or less; about 100 pM or less; or about 50 pM or less.

In some embodiments, the anti-WFS1 antibodies have a modified glycosylation pattern. In some applications, modification to remove undesirable glycosylation sites may be useful, or e.g., removal of a fucose moiety to increase antibody dependent cellular cytotoxicity (ADCC) function (see, Shield et al., J. Biol. Chem., 2002, 277, 26733). In other applications, removal of N-glycosylation site may reduce undesirable immune reactions against the therapeutic antibodies or increase affinities of the antibodies. In yet other applications, modification of galactosylation can be made in order to modify complement dependent cytotoxicity (CDC).

In any of the embodiments described herein, the WFS1 agonist is an example of a Type 2 diabetes therapeutic agent.

The present disclosure also provides compositions comprising a combination of an antibody or antigen-binding fragment thereof and a Type 2 diabetes therapeutic agent.

In some embodiments, the T2D therapeutic agents include but are not limited to an alpha-glucosidase inhibitor, a biguanide, a bile acid sequestrant, a dopamine-2 agonist, a Dipeptidyl Peptidase-4 (DPP-4) inhibitor, a meglitinide, a Sodium-Glucose Cotransporter-2 (SGLT2) inhibitor, a sulfonylurea, a thiazolidinedione (TZD), a Glucagon-Like Peptide-1 (GLP-1) receptor agonist, an insulin, an insulin sensitizer, a Proprotein Convertase Subtilisin/Kexin Type 9 (PCSK9) inhibitor, and an agent that preserves beta cell function and survival, or any combination thereof. In some embodiments, the T2D therapeutic agents include but are not limited to a Hepatocyte Nuclear Factor 4 Alpha (HNF4A) agonist.

Examples of alpha-glucosidase inhibitors include but are not limited to acarbose, miglitol, and voglibose, or any combination thereof.

Examples of biguanides include but are not limited to metformin, phenformin, and buformin, or any combination thereof.

Examples of bile acid sequestrants include but are not limited to cholestyramine, colesevelam, and colestipol, or any combination thereof.

Examples of dopamine-2 agonists include but are not limited to bromocriptine, pergolide, cabergoline, pramipexole, ropinirole, and rotigotine, or any combination thereof.

Examples of DPP-4 inhibitors include but are not limited to alogliptin, linagliptin, saxagliptin, sitagliptin, vildagliptin, gemigliptin, anagliptin, teneligliptin, trelagliptin, omarigliptin, evogliptin, gosogliptin, dutogliptin, neogliptin, retagliptin, denagliptin, cofrogliptin, fotagliptin, prusogliptin, and cetagliptin, or any combination thereof.

Examples of meglitinides include but are not limited to nateglinide, repaglinide, and mitiglinide, or any combination thereof.

Examples of SGLT2 inhibitors include but are not limited to canagliflozin, dapagliflozin, empagliflozin, bexagliflozin, enavogliflozin, ertugliflozin, henagliflozin, pragliflozin, luseogliflozin, remogliflozin, rongliflozin, sergliflozin, sotagliflozin, tofogliflozin, and velagliflozin, or any combination thereof.

Examples of sulfonylureas include but are not limited to glimepiride, glipizide, glyburide, acetohexamide, carbutamide, chlorpropamide, glycyclamide, metahexamide, tolazamide, tolbutamide, glibornuride, gliclazide, gliquidone, glisoxepide, glyhexamide, phenbutamide, tolcyclamide, and glyclopyramide, or any combination thereof.

Examples of TZDs include but are not limited to rosiglitazone, pioglitazone, lobeglitazone, 5-((4-(2-(methyl-2-pyridinylamino) ethoxy)phenyl)methyl)-2,4-thiazolidinedione, troglitazone, ciglitazone, WAY-120,744, englitazone, AD 5075, and darglitazone, or any combination thereof.

Examples of GLP-1 receptor agonists include but are not limited to exenatide, liraglutide, dulaglutide, lixisenatide, semaglutide, tirzepatide, and albiglutide, or any combination thereof. The GLP-1 receptor agonist can be any pharmacological entity with any agonist activity to the GLP-1 receptor regardless of whether or not the GLP-1 receptor agonist has other pharmacological properties related to any other pathway or target. In some embodiments, the GLP-1 receptor agonist comprises a dual GLP-1/Glucose-dependent Insulinotropic Polypeptide (GIP) (GLP-1/GIP) agonist, such as tirzepatide, maridebart cafraglutide (MariTide), or NNC0090-2746 (RG7697). In some embodiments, the GLP-1 receptor agonist comprises a dual GLP-1/amylin receptor agonist, such as zenagamtide, CagriSema (combination of semaglutide and cagrilintide), or ASC36/ASC35. In some embodiments, the GLP-1 receptor agonist comprises a triple GLP-1/GIP/glucagon receptor (GCGR) (GLP-1/GIP/GCGR) agonist, such as retatrutide (LY3437943). In some embodiments, the GLP-1 receptor agonist comprises a dual GLP-1/GCGR agonist, such as survodutide (BI 456906) or mazdutide (IBI362).

Examples of insulins include but are not limited to insulin glulisine, insulin lispro, insulin aspart, insulin glargine, insulin detemir, and insulin isophane, or any combination thereof.

Examples of insulin sensitizers include but are not limited to metformin and a TZD, or a combination thereof.

Examples of PCSK9 inhibitors include but are not limited to alirocumab, evolocumab, inclisiran, lupin peptide, resveratrol, lycopene, and eugenol, or any combination thereof.

Examples of HNF4A agonists include but are not limited to alverine, N-trans caffeoyltyramine (NCT), HNF4A protein, and a small activating ribonucleic acid (saRNA). Examples of saRNA molecules include but are not limited to those disclosed in U.S. Patent Application Publication No. US 2022/0090074 and Huang et al., Mol. Ther. Nucl. Acids, 2020, 19, 361-370.

In some embodiments, the Type 2 diabetes therapeutic agent can be combined with the WFS1 agonist.

In some embodiments, the dose of the Type 2 diabetes therapeutic agents can be decreased by about 10%, by about 20%, by about 30%, by about 40%, by about 50%, by about 60%, by about 70%, by about 80%, or by about 90% for subjects that are heterozygous for a WFS1 variant nucleic acid molecule or WFS1 reference (i.e., a less than the standard dosage amount) compared to subjects that are homozygous for a WFS1 variant nucleic acid molecule (who may receive a standard dosage amount). In some embodiments, the dose of the Type 2 diabetes therapeutic agents can be decreased by about 10%, by about 20%, by about 30%, by about 40%, or by about 50%. In some embodiments, the dose of the Type 2 diabetes therapeutic agents can be decreased by about 10%, by about 20%, by about 30%, by about 40%, by about 50%, by about 60%, by about 70%, by about 80%, or by about 90% for subjects that are WFS1 reference compared to subjects that are heterozygous for a WFS1 variant nucleic acid molecule. In addition, subjects that are heterozygous for a WFS1 variant nucleic acid molecule or WFS1 reference can be administered the Type 2 diabetes therapeutic agents less frequently compared to subjects that are homozygous for the WFS1 variant nucleic acid molecule.

Administration of the Type 2 diabetes therapeutic agents and/or WFS1 agonists can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more.

Administration of the Type 2 diabetes therapeutic agents and/or WFS1 agonists can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients, or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” as used herein, refer to eliciting the desired biological response, such as a therapeutic effect. The terms “prevent”, “preventing”, and “prevention” as used herein, refer to eliciting the desired biological response, such as a prophylactic effect. In some embodiments, a therapeutic effect comprises one or more of a decrease/reduction in Type 2 diabetes, a decrease/reduction in the severity of Type 2 diabetes (such as, for example, a reduction or inhibition of development of Type 2 diabetes), a decrease/reduction in symptoms and disease-related effects, delaying the onset of symptoms and disease-related effects, reducing the severity of symptoms of disease-related effects, reducing the number of symptoms and disease-related effects, reducing the latency of symptoms and disease-related effects, an amelioration of symptoms and disease-related effects, reducing secondary symptoms, reducing secondary infections, preventing relapse to Type 2 diabetes, decreasing the number or frequency of relapse episodes, increasing latency between symptomatic episodes, increasing time to sustained progression, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of Type 2 diabetes development/progression (such as, for example, a complete or partial avoidance/inhibition or a delay), and an increased survival time of the affected host animal, following administration of a therapeutic protocol. Treatment of Type 2 diabetes encompasses the treatment of a subject already diagnosed as having any form of Type 2 diabetes at any clinical stage or manifestation, the delay of the onset or evolution or aggravation or deterioration of the symptoms or signs of Type 2 diabetes, and/or preventing and/or reducing the severity of Type 2 diabetes.

In some embodiments, the WFS1 agonist and the Type 2 diabetes therapeutic agent are disposed within a pharmaceutical composition. In some embodiments, the WFS1 agonist is disposed within a first pharmaceutical composition and the Type 2 diabetes therapeutic agent is disposed within a second pharmaceutical composition. In some embodiments, the first pharmaceutical composition and the second pharmaceutical composition are administered simultaneously. In some embodiments, the first pharmaceutical composition is administered before the second pharmaceutical composition. In some embodiments, the first pharmaceutical composition is administered after the second pharmaceutical composition.

The present disclosure also provides methods of identifying a subject having an increased risk of developing Type 2 diabetes. In some embodiments, the method comprises determining or having determined in a biological sample obtained from the subject the presence or absence of a WFS1 variant nucleic acid molecule (such as a genomic nucleic acid molecule, mRNA molecule, and/or cDNA molecule) encoding a WFS1 variant gain-of-function polypeptide. When the subject lacks a WFS1 variant nucleic acid molecule encoding a WFS1 variant gain-of-function polypeptide (i.e., the subject is genotypically categorized as WFS1 reference), then the subject has an increased risk of developing Type 2 diabetes. When the subject has a WFS1 variant nucleic acid molecule encoding a WFS1 variant gain-of-function polypeptide (i.e., the subject is heterozygous or homozygous for a WFS1 variant nucleic acid molecule), then the subject has a decreased risk of developing Type 2 diabetes.

The present disclosure also provides methods of identifying a subject having increased odds of slowing or preventing the progression of prediabetes to Type 2 diabetes. In some embodiments, the method comprises determining or having determined in a biological sample obtained from the subject the presence or absence of a WFS1 variant nucleic acid molecule (such as a genomic nucleic acid molecule, mRNA molecule, and/or cDNA molecule) encoding a WFS1 variant gain-of-function polypeptide. When the subject lacks a WFS1 variant nucleic acid molecule encoding a WFS1 variant gain-of-function polypeptide (i.e., the subject is genotypically categorized as WFS1 reference), then the subject has decreased odds of slowing or preventing the progression of prediabetes to Type 2 diabetes. When the subject has a WFS1 variant nucleic acid molecule encoding a WFS1 variant gain-of-function polypeptide (i.e., the subject is heterozygous or homozygous for a WFS1 variant nucleic acid molecule), then the subject has increased odds of slowing or preventing the progression of prediabetes to Type 2 diabetes.

The present disclosure also provides methods of identifying a subject having increased odds of slowing or preventing progressive beta cell dysfunction. In some embodiments, the method comprises determining or having determined in a biological sample obtained from the subject the presence or absence of a WFS1 variant nucleic acid molecule (such as a genomic nucleic acid molecule, mRNA molecule, and/or cDNA molecule) encoding a WFS1 variant gain-of-function polypeptide. When the subject lacks a WFS1 variant nucleic acid molecule encoding a WFS1 variant gain-of-function polypeptide (i.e., the subject is genotypically categorized as WFS1 reference), then the subject has decreased odds of slowing or preventing progressive beta cell dysfunction. When the subject has a WFS1 variant nucleic acid molecule encoding a WFS1 variant gain-of-function polypeptide (i.e., the subject is heterozygous or homozygous for a WFS1 variant nucleic acid molecule), then the subject has increased odds of slowing or preventing progressive beta cell dysfunction.

In any of the methods described herein, the WFS1 variant nucleic acid molecule is any of the WFS1 variant nucleic acid molecules described herein. In some embodiments, the WFS1 variant nucleic acid molecule comprises a splice-site variant, a stop-gain variant, a start-loss variant, a stop-loss variant, a frameshift variant, a missense variant, or an in-frame indel variant. In some embodiments, the WFS1 variant nucleic acid molecule encodes WFS1 Glu794Lys. In some embodiments, the WFS1 variant nucleic acid molecule is a WFS1 variant genomic nucleic acid molecule that comprises the genetic variation 4:6302175:G:A, or is an mRNA molecule produced therefrom, or is a cDNA molecule produced from the mRNA molecule. In any of the methods described herein, the WFS1 agonist comprises any of the WFS1 agonists described herein.

Having a single copy of a WFS1 variant nucleic acid molecule is more protective of a subject from developing Type 2 diabetes than having no copies of a WFS1 variant nucleic acid molecule. Without intending to be limited to any particular theory or mechanism of action, it is believed that a single copy of a WFS1 variant nucleic acid molecule (i.e., heterozygous for a WFS1 variant nucleic acid molecule) is protective of a subject from developing Type 2 diabetes and it is also believed that having two copies of a WFS1 variant nucleic acid molecule (i.e., homozygous for a WFS1 variant nucleic acid molecule) may be more protective of a subject from developing Type 2 diabetes, relative to a subject with a single copy. Thus, in some embodiments, a single copy of a WFS1 variant nucleic acid molecule may not be completely protective, but instead, may be partially or incompletely protective of a subject from developing Type 2 diabetes. While not desiring to be bound by any particular theory, there may be additional factors or molecules involved in the development of Type 2 diabetes that are still present in a subject having a single copy of a WFS1 variant nucleic acid molecule, thus resulting in less than complete protection from the development of Type 2 diabetes.

Determining whether a subject has a WFS1 variant nucleic acid molecule in a biological sample from a subject and/or determining whether a subject has a WFS1 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the nucleic acid molecule can be present within a cell obtained from the subject.

In some embodiments, when a subject is identified as having an increased risk of developing Type 2 diabetes, the subject is administered a Type 2 diabetes therapeutic agent and/or a WFS1 agonist, as described herein. For example, when the subject is WFS1 reference, and therefore has an increased risk of developing Type 2 diabetes, the subject is administered a Type 2 diabetes therapeutic agent in an amount that is the same as or less than a standard dosage amount and/or is administered a WFS1 agonist. In some embodiments, when the subject is WFS1 reference, and therefore has an increased risk of developing Type 2 diabetes, the subject is administered a Type 2 diabetes therapeutic agent in an amount that is the same as or less than a standard dosage amount and is administered a WFS1 agonist. In some embodiments, when the subject is heterozygous for a WFS1 variant nucleic acid molecule, the subject is administered the Type 2 diabetes therapeutic agent in an amount that is the same as or less than a standard dosage amount and/or is administered a WFS1 agonist. In some embodiments, when the subject is heterozygous for a WFS1 variant nucleic acid molecule, the subject is administered the Type 2 diabetes therapeutic agent in an amount that is the same as or less than a standard dosage amount and is administered a WFS1 agonist. In some embodiments, when the subject is homozygous for a WFS1 variant nucleic acid molecule, the subject is administered a Type 2 diabetes therapeutic agent in an amount that is the same as or less than a standard dosage amount and/or a WFS1 agonist. In some embodiments, when the subject is homozygous for a WFS1 variant nucleic acid molecule, the subject is administered a Type 2 diabetes therapeutic agent in an amount that is the same as or less than a standard dosage amount and a WFS1 agonist. In some embodiments, the subject is WFS1 reference. In some embodiments, the subject is heterozygous for a WFS1 variant nucleic acid molecule. In some embodiments, the subject is homozygous for a WFS1 variant nucleic acid molecule.

The present disclosure also provides methods of detecting the presence or absence of a WFS1 variant nucleic acid molecule (i.e., a genomic nucleic acid molecule, an mRNA molecule, or a cDNA molecule produced from an mRNA molecule) in a biological sample from a subject. It is understood that gene sequences within a population and mRNA molecules encoded by such genes can vary due to polymorphisms such as single-nucleotide polymorphisms.

The biological sample can be derived from any cell, tissue, or biological fluid from the subject. The biological sample may comprise any clinically relevant tissue, such as a bone marrow sample, a tumor biopsy, a fine needle aspirate, or a sample of bodily fluid, such as blood, gingival crevicular fluid, plasma, serum, lymph, ascitic fluid, cystic fluid, or urine. In some cases, the sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any WFS1 variant nucleic acid molecule, preliminary processing designed to isolate or enrich the biological sample for the genomic DNA can be employed. A variety of techniques may be used for this purpose. When detecting the level of any WFS1 variant nucleic acid molecule, different techniques can be used enrich the biological sample with mRNA molecules. Various methods to detect the presence or level of an mRNA molecule or the presence of a particular variant genomic DNA locus can be used.

In some embodiments, detecting a WFS1 variant nucleic acid molecule in a subject comprises performing a sequence analysis on a biological sample obtained from the subject to determine whether a WFS1 genomic nucleic acid molecule in the biological sample, and/or a WFS1 mRNA molecule in the biological sample, and/or a WFS1 cDNA molecule produced from an mRNA molecule in the biological sample, is present in the sample. In some embodiments, the methods detect the WFS1 variant genomic nucleic acid molecule that comprises the genetic variation 4:6302175:G:A, or an mRNA molecule produced therefrom, or a cDNA molecule produced from the mRNA molecule.

In some embodiments, the methods of detecting the presence or absence of a WFS1 variant nucleic acid molecule (such as, for example, a genomic nucleic acid molecule, an mRNA molecule, and/or a cDNA molecule produced from an mRNA molecule) in a subject comprise performing an assay on a biological sample obtained from the subject. The assay determines whether a nucleic acid molecule in the biological sample comprises a particular nucleotide sequence.

In some embodiments, the biological sample comprises a cell or cell lysate. Such methods can further comprise, for example, obtaining a biological sample from the subject comprising a WFS1 genomic nucleic acid molecule or mRNA molecule, and if mRNA, optionally reverse transcribing the mRNA into cDNA. Such assays can comprise, for example determining the identity of these positions of the particular WFS1 nucleic acid molecule. In some embodiments, the method is an in vitro method.

In some embodiments, the determining step, detecting step, or sequence analysis comprises sequencing at least a portion of the nucleotide sequence of the WFS1 genomic nucleic acid molecule, the WFS1 mRNA molecule, or the WFS1 cDNA molecule in the biological sample that comprises a genetic variation compared to the corresponding WFS1 reference molecule. In some embodiments, the sequenced portion comprises one or more variations that cause a gain-of-function (partial or complete) or are predicted to cause a gain-of-function (partial or complete).

In some embodiments, the assay comprises sequencing the entire nucleic acid molecule. In some embodiments, only a WFS1 genomic nucleic acid molecule is analyzed. In some embodiments, only a WFS1 mRNA is analyzed. In some embodiments, only a WFS1 cDNA obtained from the WFS1 mRNA is analyzed.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the nucleic acid molecule in the sample is mRNA and the mRNA is reverse-transcribed into a cDNA prior to the amplifying step. In some embodiments, the nucleic acid molecule is present within a cell obtained from the subject.

In some embodiments, the assay comprises contacting the biological sample with a primer or probe, such as an alteration-specific primer or alteration-specific probe, that specifically hybridizes to a WFS1 variant genomic sequence, variant mRNA sequence, or variant cDNA sequence and not the corresponding WFS1 reference sequence under stringent conditions and determining whether hybridization has occurred.

In some embodiments, the determining step, detecting step, or sequence analysis comprises: a) amplifying at least a portion of the WFS1 nucleic acid molecule that encodes the WFS1 polypeptide; b) labeling the amplified nucleic acid molecule with a detectable label; c) contacting the labeled nucleic acid molecule with a support comprising an alteration-specific probe; and d) detecting the detectable label.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising a WFS1 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence-based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6× sodium chloride/sodium citrate (SSC) at about 45 °C, followed by a wash of 2×SSC at 50 °C, are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30 °C for short probes (such as, for example, 10 to 50 nucleotides) and at least about 60 °C for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, such isolated nucleic acid molecules hybridize to WFS1 variant nucleic acid molecules (such as genomic nucleic acid molecules, mRNA molecules, and/or cDNA molecules) under stringent conditions. Such nucleic acid molecules can be used, for example, as probes, primers, alteration-specific probes, or alteration-specific primers as described or exemplified herein, and include, without limitation primers, probes, antisense RNAs, shRNAs, and siRNAs, each of which is described in more detail elsewhere herein and can be used in any of the methods described herein.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to WFS1 variant nucleic acid molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the WFS1 variant nucleic acid molecules disclosed herein. The primers described herein can be used to amplify any WFS1 variant nucleic acid molecule, or a fragment thereof.

In the context of the disclosure “specifically hybridizes” means that the probe or primer (such as, for example, the alteration-specific probe or alteration-specific primer) does not hybridize to a nucleic acid sequence encoding a WFS1 reference genomic nucleic acid molecule, a WFS1 reference mRNA molecule, and/or a WFS1 reference cDNA molecule.

In some embodiments, the probes (such as, for example, an alteration-specific probe) comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules disclosed herein can comprise RNA, DNA, or both RNA and DNA. The isolated nucleic acid molecules can also be linked or fused to a heterologous nucleic acid sequence, such as in a vector, or a heterologous label. For example, the isolated nucleic acid molecules disclosed herein can be within a vector or as an exogenous donor sequence comprising the isolated nucleic acid molecule and a heterologous nucleic acid sequence. The isolated nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3×FLAG, 6×his or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

The present disclosure also provides Type 2 diabetes therapeutic agents for use in the treatment or prevention of Type 2 diabetes, slowing or preventing the progression of prediabetes to Type 2 diabetes, or slowing or preventing progressive beta cell dysfunction in a subject having a WFS1 variant nucleic acid molecule. The Type 2 diabetes is any of the Type 2 diabetes described herein. The Type 2 diabetes therapeutic agent is any of the Type 2 diabetes therapeutic agents described herein. The WFS1 variant nucleic acid molecule is any of the WFS1 variant nucleic acid molecules described herein.

The present disclosure also provides the use of Type 2 diabetes therapeutic agents in the preparation of a medicament for treating or preventing Type 2 diabetes, slowing or preventing the progression of prediabetes to Type 2 diabetes, or slowing or preventing progressive beta cell dysfunction in a subject having a WFS1 variant nucleic acid molecule. The Type 2 diabetes is any of the Type 2 diabetes described herein. The Type 2 diabetes therapeutic agent is any of the Type 2 diabetes therapeutic agents described herein. The WFS1 variant nucleic acid molecule is any of the WFS1 variant nucleic acid molecules described herein.

The present disclosure also provides WFS1 agonists for use in the treatment or prevention of Type 2 diabetes, slowing or preventing the progression of prediabetes to Type 2 diabetes, or slowing or preventing progressive beta cell dysfunction in a subject that is WFS1 reference or in a subject that is heterozygous for a WFS1 variant nucleic acid molecule encoding a WFS1 variant gain-of-function polypeptide. The Type 2 diabetes is any of the Type 2 diabetes described herein. The WFS1 agonist is any of the WFS1 agonists described herein. The WFS1 variant nucleic acid molecule is any of the WFS1 variant nucleic acid molecules described herein. In some embodiments, the WFS1 agonist is used in combination with any one or more of the Type 2 diabetes therapeutic agents described herein.

The present disclosure also provides the use of WFS1 agonists in the preparation of a medicament for treating or preventing Type 2 diabetes, slowing or preventing the progression of prediabetes to Type 2 diabetes, or slowing or preventing progressive beta cell dysfunction in a subject that is WFS1 reference or in a subject that is heterozygous for a WFS1 variant nucleic acid molecule encoding a WFS1 variant gain-of-function polypeptide. The Type 2 diabetes is any of the Type 2 diabetes described herein. The WFS1 agonist is any of the WFS1 agonists described herein. The WFS1 variant nucleic acid molecule is any of the WFS1 variant nucleic acid molecules described herein. In some embodiments, the WFS1 agonist is used in combination with any one or more of the Type 2 diabetes therapeutic agents described herein.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in °C or is at ambient temperature, and pressure is at or near atmospheric.

### EXAMPLES

### Example 1: Exome/Genome Wide Association Study of T2D

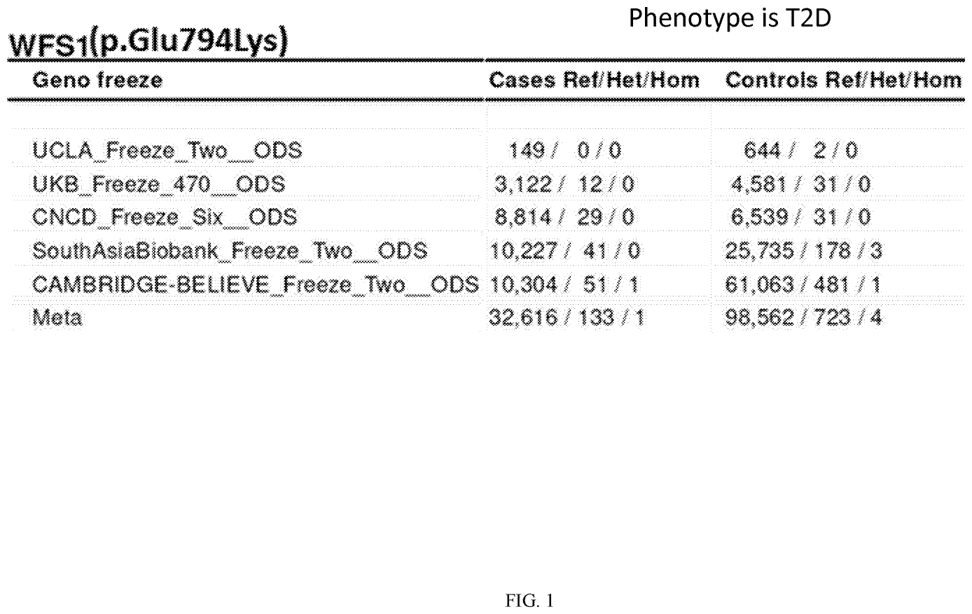

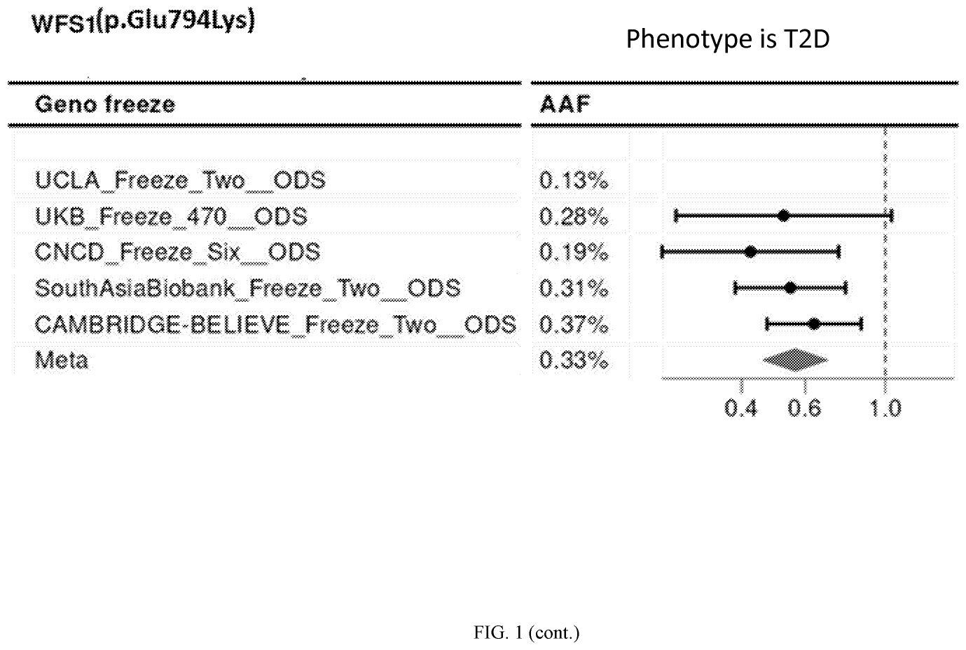

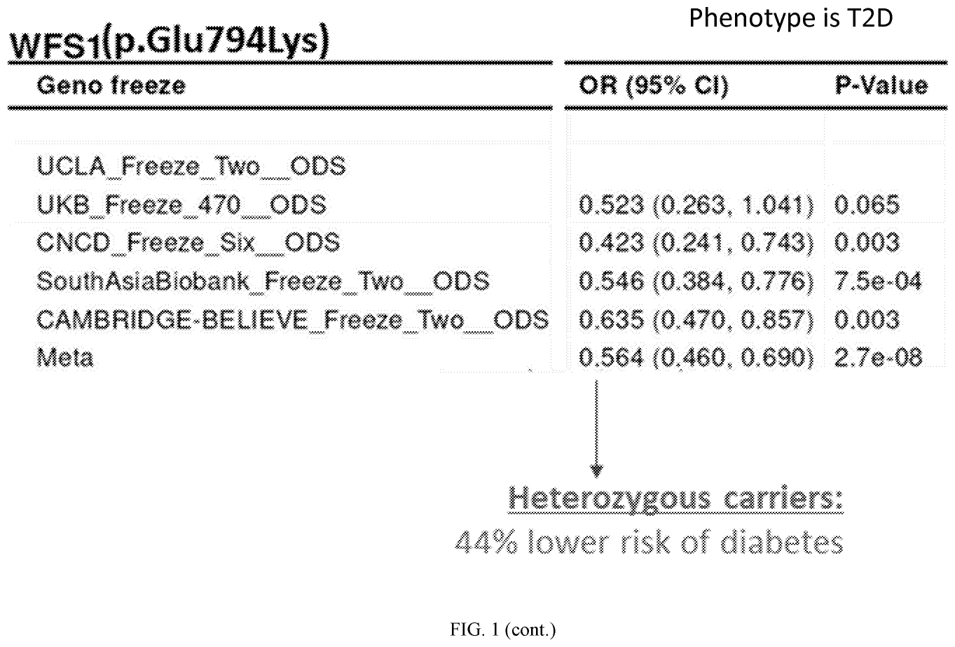

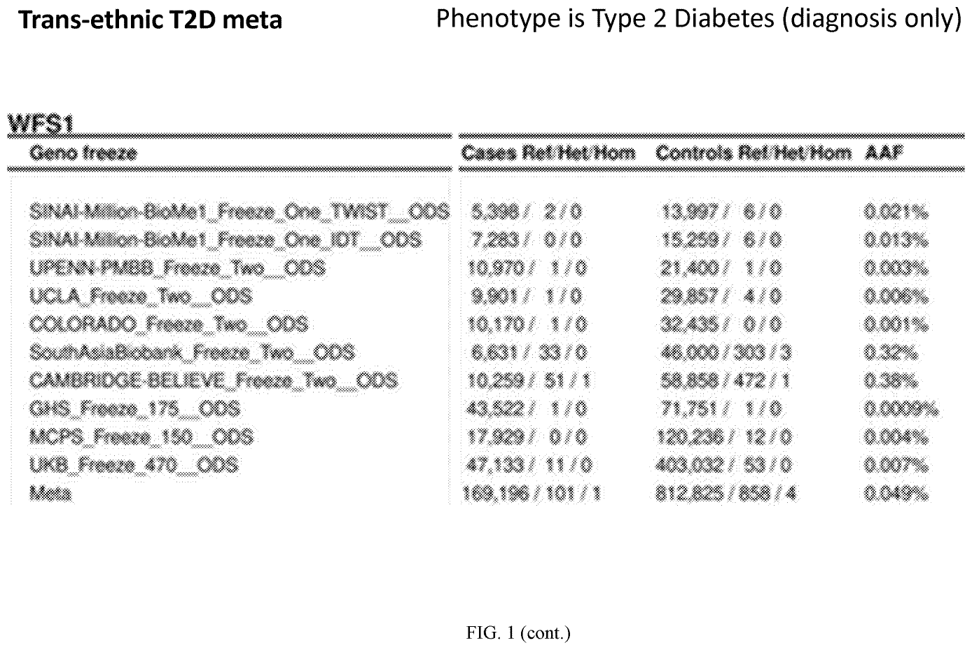

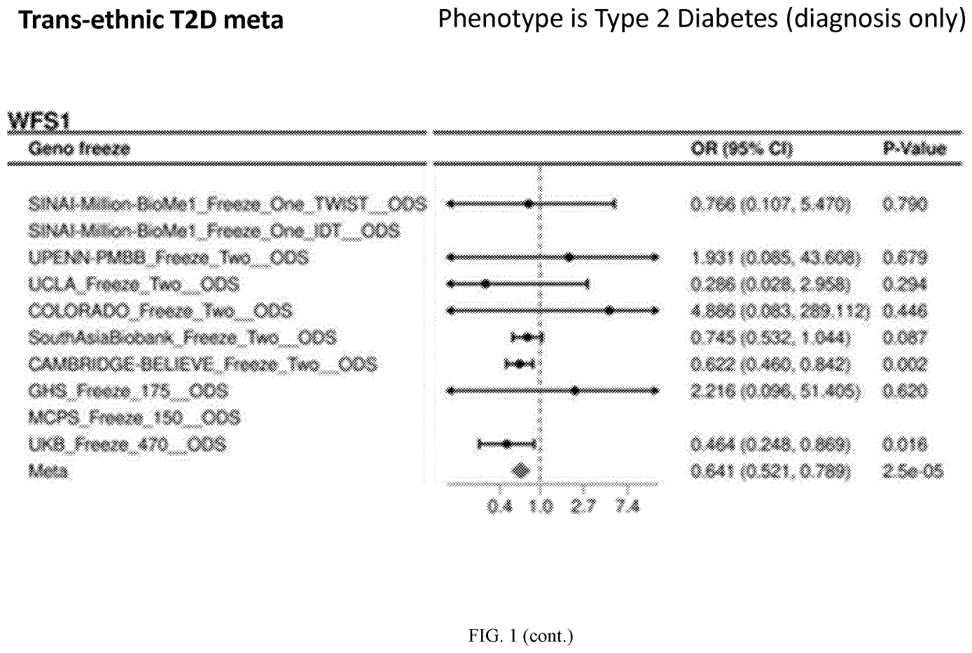

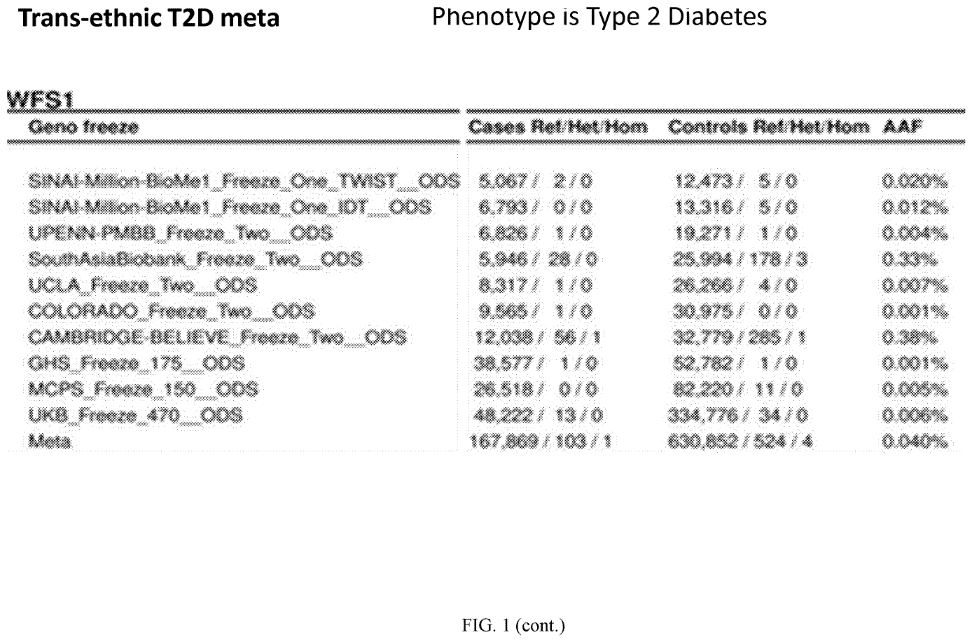

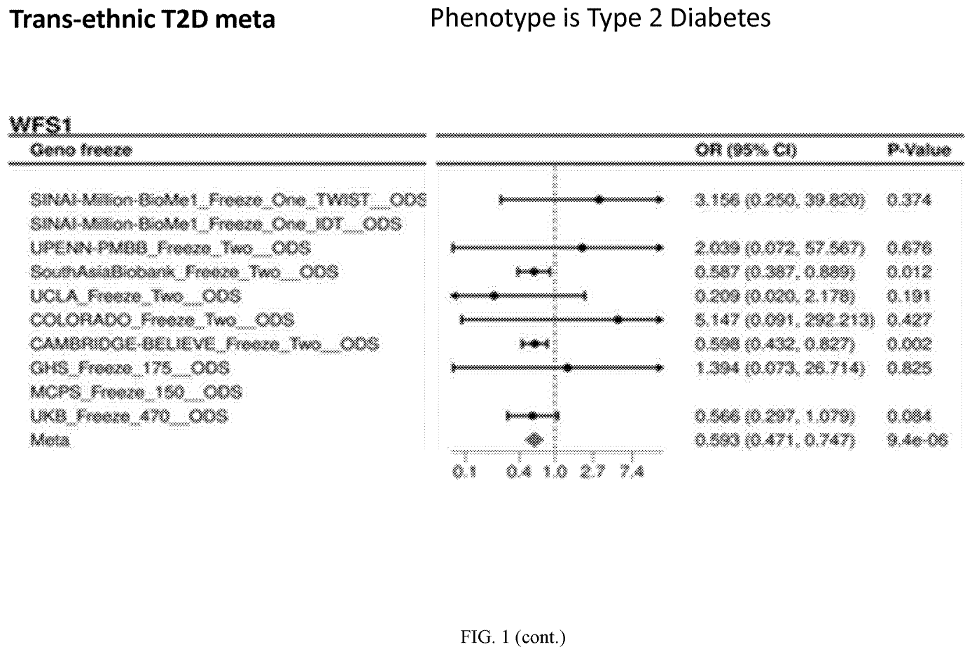

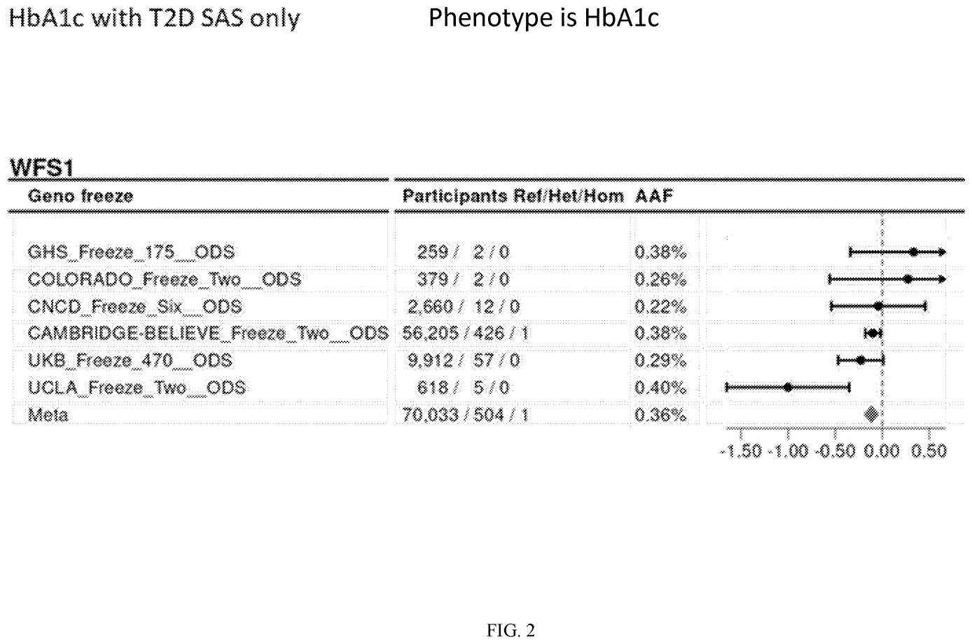

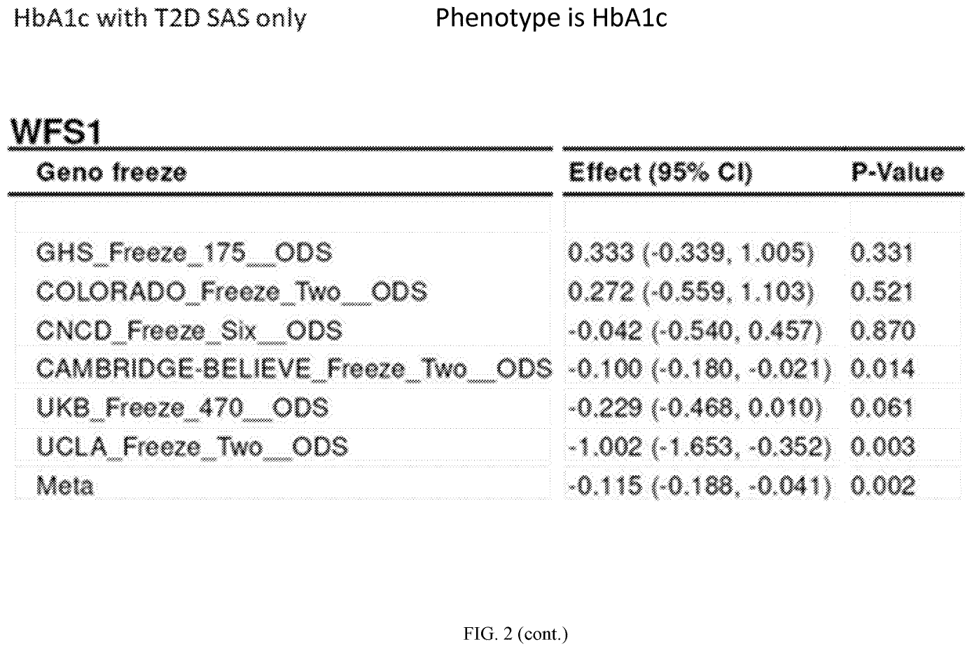

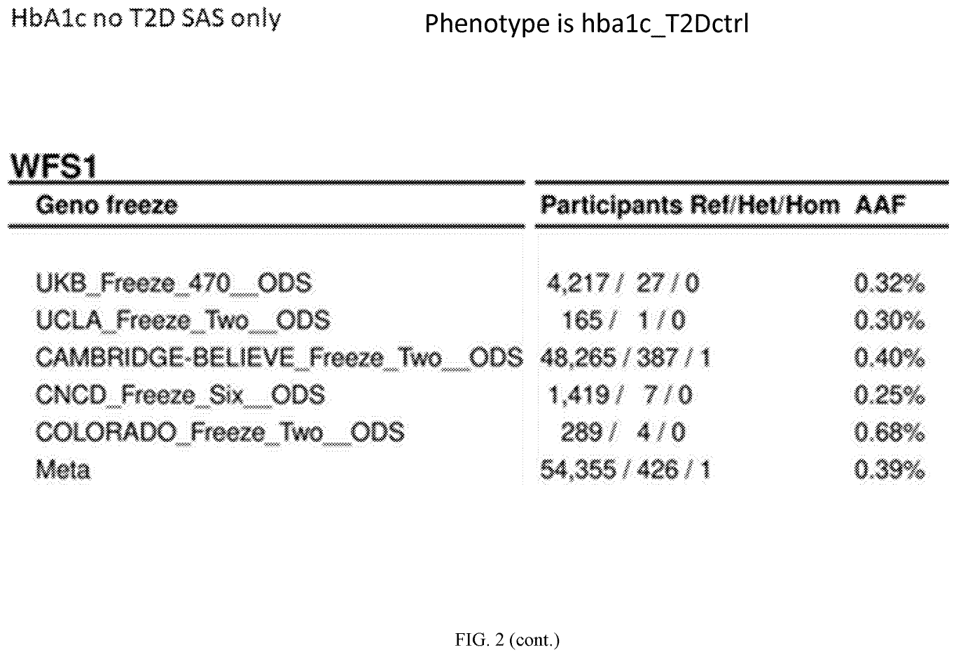

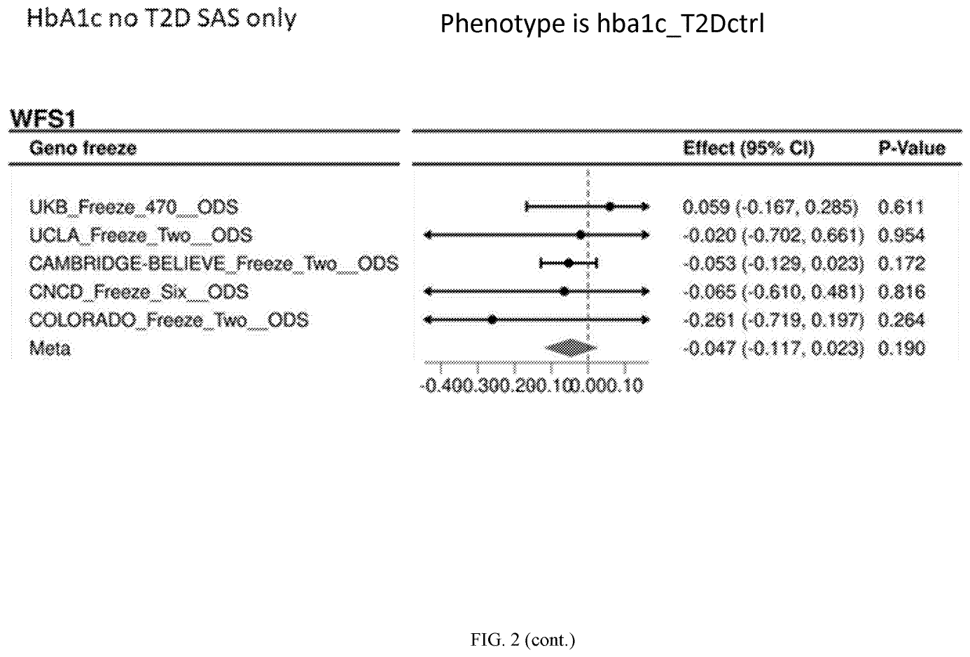

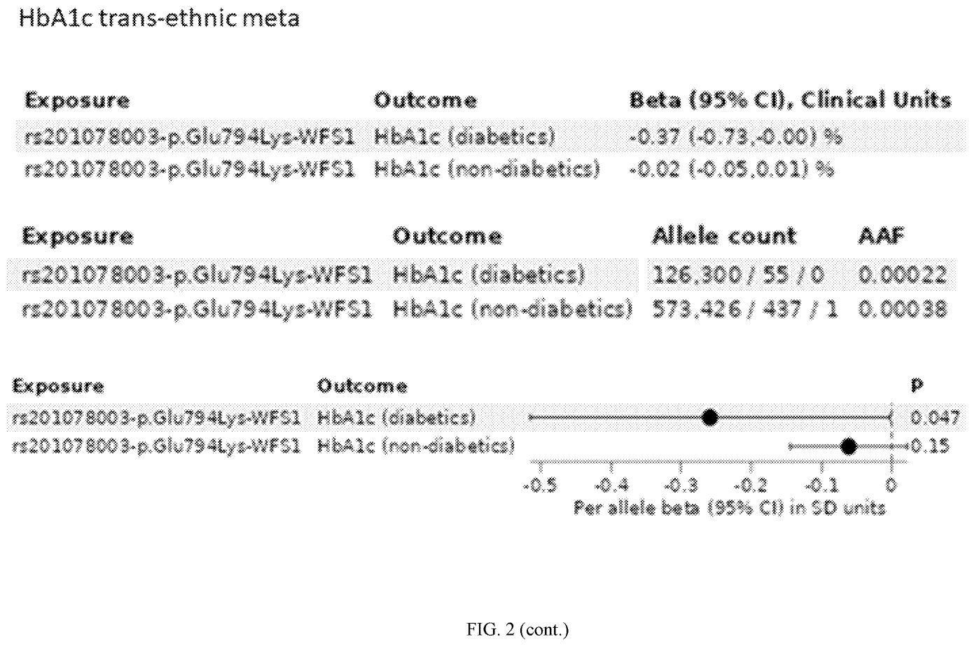

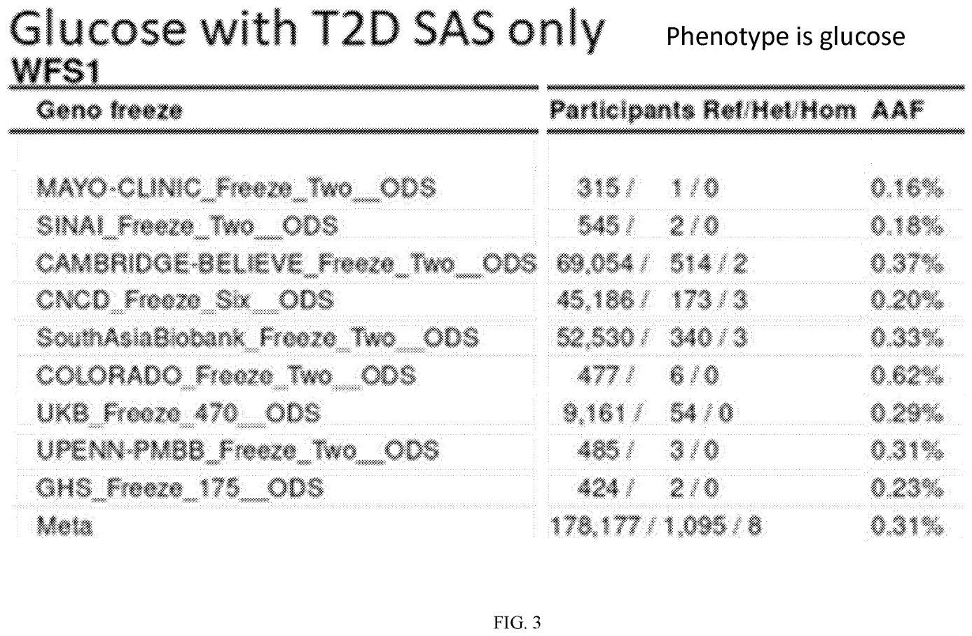

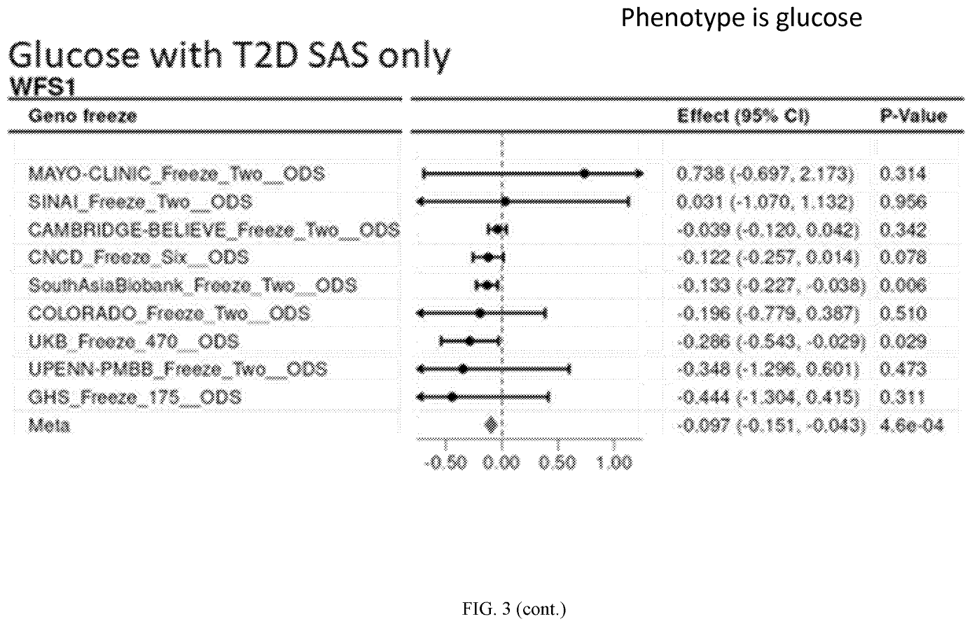

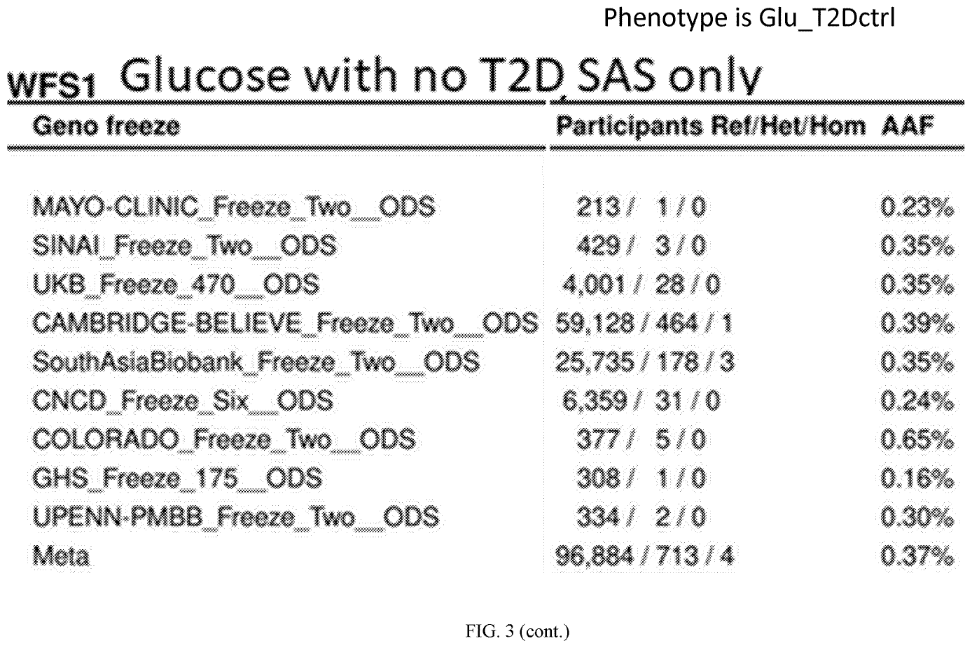

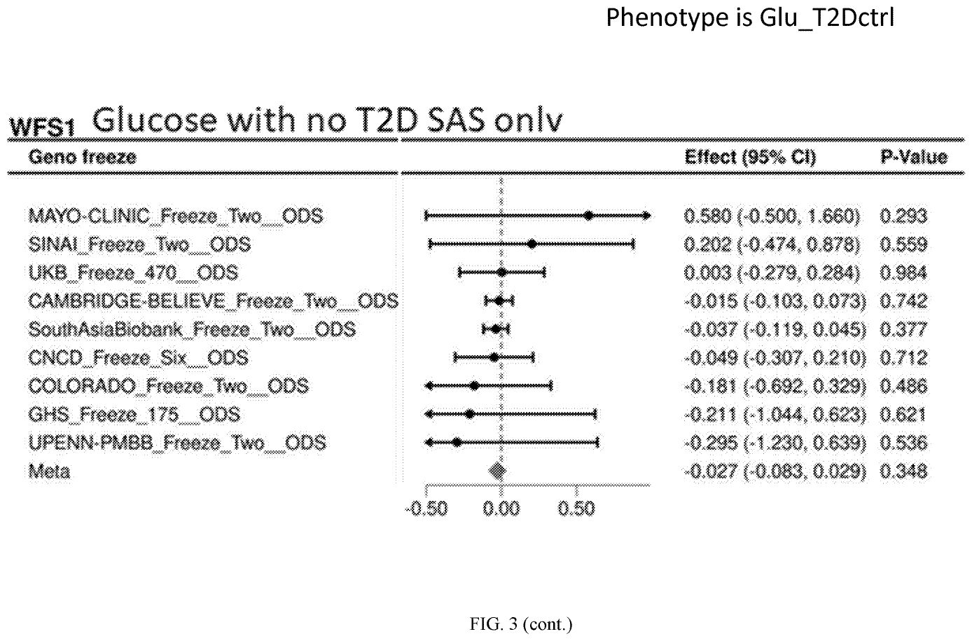

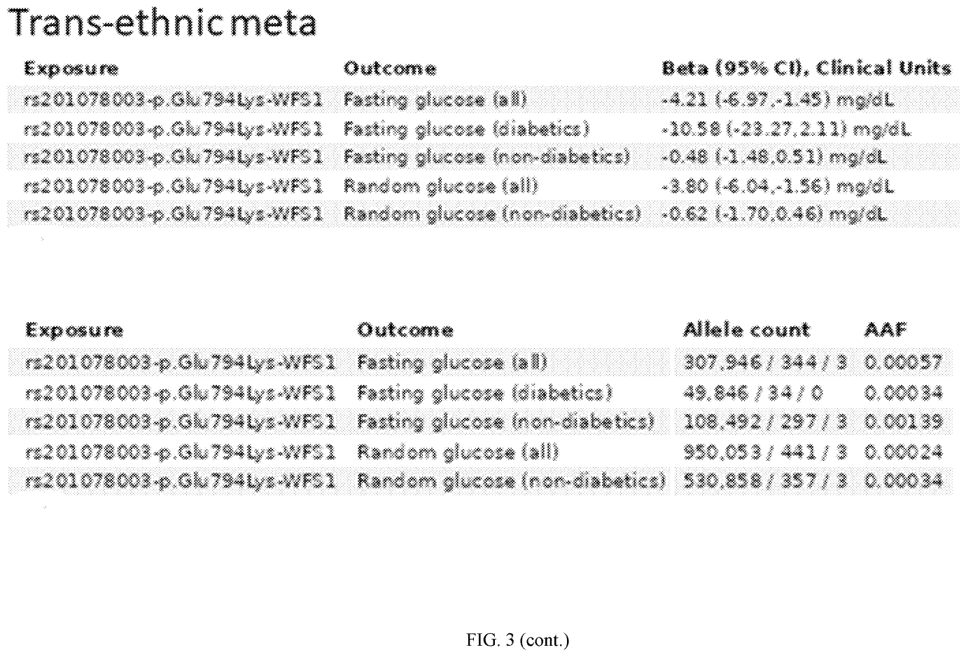

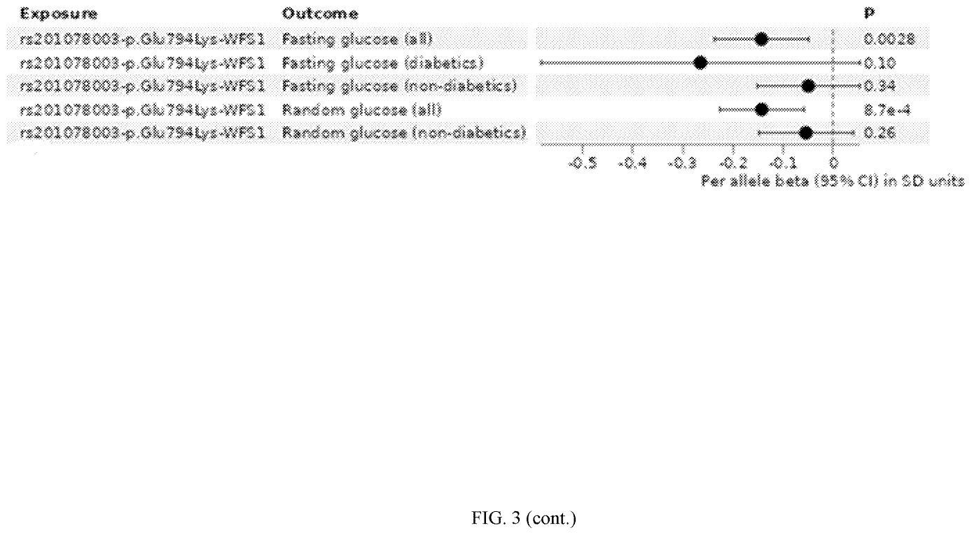

Exome/genome wide association study of T2D was performed in South Asians. A total of 32,752 T2D cases and 99,293 controls were included in the analysis and controlled for sex, age and genetics Principal components using REGEINE. A missense variant 4:6302175:G: A (p.Glu794Lys) in WFS1 associated with protection from T2D in South Asians (effect 0.56, p=2.7e-08) (see, FIG. 1, FIG. 2, and FIG. 3). The allele frequency of variant 4:6302175:G:A is 0.33% in South Asian population compared Non-finish Europeans (0.0009%) (Table 1).

TABLE 1
Frequency of Missense variant 4:6302175:G:A (p.Glu794Lys) enriched
in South Asians compared to populations from other ancestries.

| Ancestry | Alternate Allele Frequency |
|---|---|
| All | 0.00009672 |
| African | 0.000004544 |
| West Africa | 0.000004877 |
| South Africa | 0.000002122 |
| South Asia | 0.002 |
| Indian | 0.003 |
| Pakistani | 0.002 |
| East Asia | 0.00003488 |
| South-East Asia | 0.0002412 |
| European | 0.000009841 |
| Finland | 0.00002326 |
| Central Europe | 0.00002665 |
| Indigenous American | 0.000027 |
| Middle East | 0.0001011 |

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having Type 2 diabetes or at risk of developing Type 2 diabetes, the method comprising administering a Wolfram Syndrome 1 (WFS1) agonist to the subject.

2. The method of claim 1, wherein the subject is WFS1 reference.

3. The method of claim 1, wherein the subject is heterozygous for a WFS1 variant nucleic acid molecule encoding a WFS1 variant gain-of-function polypeptide.

4. The method of claim 1, wherein the subject is homozygous for a WFS1 variant nucleic acid molecule encoding a WFS1 variant gain-of-function polypeptide.

5. The method of claim 3, wherein the WFS1 variant nucleic acid molecule comprises a splice-site variant, a stop-gain variant, a start-loss variant, a stop-loss variant, a frameshift variant, a missense variant, or an in-frame indel variant.

6. The method of claim 3, wherein the WFS1 variant nucleic acid molecule encodes WFS1 Glu794Lys.

7. The method of claim 3, wherein the WFS1 variant nucleic acid molecule comprises the genetic variation 4:6302175:G:A.

8. The method of claim 1, wherein the WFS1 agonist comprises a wolframin protein.

9. The method of claim 1, wherein the subject is also administered a Type 2 diabetes therapeutic agent.

10. The method of claim 9, wherein the Type 2 diabetes therapeutic agent comprises an alpha-glucosidase inhibitor, a biguanide, a bile acid sequestrant, a dopamine-2 agonist, a Dipeptidyl Peptidase-4 (DPP-4) inhibitor, a meglitinide, a Sodium-Glucose Cotransporter-2 (SGLT2) inhibitor, a sulfonylurea, a thiazolidinedione (TZD), a Glucagon-Like Peptide-1 (GLP-1) receptor agonist, an insulin, an insulin sensitizer, a Proprotein Convertase Subtilisin/Kexin Type 9 (PCSK9) inhibitor, or an agent that preserves beta cell function and survival, or any combination thereof.

11. The method of claim 10, wherein:
the alpha-glucosidase inhibitor comprises acarbose, miglitol, or voglibose, or any combination thereof;
the biguanide comprises metformin, phenformin, or buformin, or any combination thereof;
the bile acid sequestrant comprises cholestyramine, colesevelam, or colestipol, or any combination thereof;
the dopamine-2 agonist comprises bromocriptine, pergolide, cabergoline, pramipexole, ropinirole, or rotigotine, or any combination thereof;
the DPP-4 inhibitor comprises alogliptin, linagliptin, saxagliptin, sitagliptin, vildagliptin, gemigliptin, anagliptin, teneligliptin, trelagliptin, omarigliptin, evogliptin, gosogliptin, dutogliptin, neogliptin, retagliptin, denagliptin, cofrogliptin, fotagliptin, prusogliptin, or cetagliptin, or any combination thereof;
the meglitinide comprises nateglinide, repaglinide, or mitiglinide, or any combination thereof;
the SGLT2 inhibitor comprises canagliflozin, dapagliflozin, empagliflozin, bexagliflozin, enavogliflozin, ertugliflozin, henagliflozin, pragliflozin, luseogliflozin, remogliflozin, rongliflozin, sergliflozin, sotagliflozin, tofogliflozin, or velagliflozin, or any combination thereof;
the sulfonylurea comprises glimepiride, glipizide, glyburide, acetohexamide, carbutamide, chlorpropamide, glycyclamide, metahexamide, tolazamide, tolbutamide, glibornuride, gliclazide, gliquidone, glisoxepide, glyhexamide, phenbutamide, tolcyclamide, or glyclopyramide, or any combination thereof;
the TZD comprises rosiglitazone, pioglitazone, lobeglitazone, 5-((4-(2-(methyl-2-pyridinylamino) ethoxy)phenyl)methyl)-2,4-thiazolidinedione, troglitazone, ciglitazone, WAY-120,744, englitazone, AD 5075, or darglitazone, or any combination thereof;
the GLP-1 receptor agonist comprises exenatide, liraglutide, dulaglutide, lixisenatide, semaglutide, tirzepatide, maridebart cafraglutide, NNC0090-2746, zenagamtide, a combination of semaglutide and cagrilintide, ASC36/ASC35, retatrutide, survodutide, mazdutide, or albiglutide, or any combination thereof;
the insulin comprises insulin glulisine, insulin lispro, insulin aspart, insulin glargine, insulin detemir, or insulin isophane, or any combination thereof; and
the PCSK9 inhibitor comprises alirocumab, evolocumab, inclisiran, lupin peptide, resveratrol, lycopene, or eugenol, or any combination thereof.

12-17. (canceled)

18. A method of treating a subject having Type 2 diabetes or at risk of developing Type 2 diabetes by administering a Type 2 diabetes therapeutic agent and/or a Wolfram Syndrome 1 (WFS1) agonist to the subject, the method comprising:
determining or having determined whether the subject has a WFS1 variant nucleic acid molecule encoding a WFS1 variant gain-of-function polypeptide, by:
obtaining or having obtained a biological sample from the subject; and
performing or having performed a sequence analysis on the biological sample to determine if the subject has a genotype comprising the WFS1 variant nucleic acid molecule; and
administering or continuing to administer the Type 2 diabetes therapeutic agent in an amount that is the same as or less than a standard dosage amount and/or the WFS1 agonist to a subject that is WFS1 reference;
administering or continuing to administer the Type 2 diabetes therapeutic agent in an amount that is the same as or less than a standard dosage amount and/or the WFS1 agonist to a subject that is heterozygous for the WFS1 variant nucleic acid molecule; or
administering or continuing to administer the Type 2 diabetes therapeutic agent in an amount that is the same as or less than a standard dosage amount and/or a WFS1 agonist to a subject that is homozygous for the WFS1 variant nucleic acid molecule;
wherein the presence of the WFS1 variant nucleic acid molecule indicates the subject has a decreased risk of developing Type 2 diabetes.

19. The method of claim 18, wherein the WFS1 variant nucleic acid molecule comprises a splice-site variant, a stop-gain variant, a start-loss variant, a stop-loss variant, a frameshift variant, a missense variant, or an in-frame indel variant.

20. The method of claim 18, wherein the WFS1 variant nucleic acid molecule encodes WFS1 Glu794Lys.

21. The method of claim 18, wherein the WFS1 variant nucleic acid molecule comprises the genetic variation 4:6302175:G:A.

22. The method of claim 18, wherein the WFS1 agonist comprises a wolframin protein.

23. The method of claim 18, wherein the Type 2 diabetes therapeutic agent comprises an alpha-glucosidase inhibitor, a biguanide, a bile acid sequestrant, a dopamine-2 agonist, a Dipeptidyl Peptidase-4 (DPP-4) inhibitor, a meglitinide, a Sodium-Glucose Cotransporter-2 (SGLT2) inhibitor, a sulfonylurea, a thiazolidinedione (TZD), a Glucagon-Like Peptide-1 (GLP-1) receptor agonist, an insulin, an insulin sensitizer, a Proprotein Convertase Subtilisin/Kexin Type 9 (PCSK9) inhibitor, or an agent that preserves beta cell function and survival, or any combination thereof.

24. The method of claim 23, wherein:
the alpha-glucosidase inhibitor comprises acarbose, miglitol, or voglibose, or any combination thereof;
the biguanide comprises metformin, phenformin, or buformin, or any combination thereof;
the bile acid sequestrant comprises cholestyramine, colesevelam, or colestipol, or any combination thereof;
the dopamine-2 agonist comprises bromocriptine, pergolide, cabergoline, pramipexole, ropinirole, or rotigotine, or any combination thereof;
the DPP-4 inhibitor comprises alogliptin, linagliptin, saxagliptin, sitagliptin, vildagliptin, gemigliptin, anagliptin, teneligliptin, trelagliptin, omarigliptin, evogliptin, gosogliptin, dutogliptin, neogliptin, retagliptin, denagliptin, cofrogliptin, fotagliptin, prusogliptin, or cetagliptin, or any combination thereof;
the meglitinide comprises nateglinide, repaglinide, or mitiglinide, or any combination thereof;
the SGLT2 inhibitor comprises canagliflozin, dapagliflozin, empagliflozin, bexagliflozin, enavogliflozin, ertugliflozin, henagliflozin, pragliflozin, luseogliflozin, remogliflozin, rongliflozin, sergliflozin, sotagliflozin, tofogliflozin, or velagliflozin, or any combination thereof;
the sulfonylurea comprises glimepiride, glipizide, glyburide, acetohexamide, carbutamide, chlorpropamide, glycyclamide, metahexamide, tolazamide, tolbutamide, glibornuride, gliclazide, gliquidone, glisoxepide, glyhexamide, phenbutamide, tolcyclamide, or glyclopyramide, or any combination thereof;
the TZD comprises rosiglitazone, pioglitazone, lobeglitazone, 5-((4-(2-(methyl-2-pyridinylamino) ethoxy)phenyl)methyl)-2,4-thiazolidinedione, troglitazone, ciglitazone, WAY-120,744, englitazone, AD 5075, or darglitazone, or any combination thereof;
the GLP-1 receptor agonist comprises exenatide, liraglutide, dulaglutide, lixisenatide, semaglutide, tirzepatide, maridebart cafraglutide, NNC0090-2746, zenagamtide, a combination of semaglutide and cagrilintide, ASC36/ASC35, retatrutide, survodutide, mazdutide, or albiglutide, or any combination thereof;
the insulin comprises insulin glulisine, insulin lispro, insulin aspart, insulin glargine, insulin detemir, or insulin isophane, or any combination thereof; and
the PCSK9 inhibitor comprises alirocumab, evolocumab, inclisiran, lupin peptide, resveratrol, lycopene, or eugenol, or any combination thereof.

25. The method of claim 18, wherein the subject is heterozygous for the WFS1 variant nucleic acid molecule, and the subject is administered or continued to be administered the Type 2 diabetes therapeutic agent in an amount that is the same as or less than a standard dosage amount and the WFS1 agonist.

26. The method of claim 18, wherein the subject is WFS1 reference, and the subject is administered or continued to be administered the Type 2 diabetes therapeutic agent in an amount that is the same as or less than a standard dosage amount and the WFS1 agonist.

27. A method of identifying a subject having an increased risk of developing Type 2 diabetes, the method comprising:
determining or having determined the presence or absence of a Wolfram Syndrome 1 (WFS1) variant nucleic acid molecule encoding a WFS1 variant gain-of-function polypeptide in a biological sample obtained from the subject;
wherein:
when the subject is WFS1 reference, then the subject has an increased risk of developing Type 2 diabetes; and
when the subject is heterozygous or homozygous for the WFS1 variant nucleic acid molecule, then the subject has a decreased risk of developing Type 2 diabetes.

28-51. (canceled)
